(19) United States
(12) Patent Application Publication
(10) Pub. No.: US 2012/0283622 A1
(43) Pub. Date: Nov. 8, 2012

(54) DERMATOLOGICAL TREATMENT DEVICE

(76) Inventor: Günther Nath, Grunwald (DE)

(21) Appl. No.: 13/508,898

(22) PCT Filed: Nov. 10, 2010
(86) PCT No.: PCT/EP2010/067192

§ 371 Date: Jul. 24, 2012

(30) Foreign Application Priority Data
Nov. 10, 2009 (DE) 20 2009 015 220.0

(51) Int. Cl. A61M 37/00

(52) U.S. Cl. 604/20

(57) ABSTRACT

The invention relates to a dermatological treatment device suitable primarily for treating nail fungus, in particular toenail fungus. The use of a photochemically active substance allows effective control of the fungus by irradiating with light at a wavelength that is relatively harmless to health. Potential technical implementations are possible for setting up the irradiation device, utilizing either a gas discharge lamp or LEDs as the light source. A shoe-shaped optical shielding housing is preferably used, being transparent in the long-wavelength portion of the visible spectrum, and absorptive in the short-wavelength, therapeutic range of the spectrum.

### TECHNICAL FIELD

The present invention describes an optical treatment device which is primarily suited for the treatment of nail fungus, in particular toenail fungus and fingernail fungus.

However, because of its flexible construction, it is also suited for the treatment of locally limited inflammation areas, for example in the case of psoriasis, neuro-dermatitis and acne.

### PRIOR ART

The U.S. Pat. No. 7,306,620 of Cumbie gives a very detailed description of the methods hitherto applied to the treatment of fungus infections, in particular also of nail fungus infections, with the aid of optical radiation. Cumbie particularly prefers the spectral range from 100 nm to 400 nm. As light sources, generally “polychromatic” emitters such as low-pressure mercury and xenon lamps are mentioned. Cumbie omits further details regarding an irradiation device suitable for the practice. However, the US patent of Cumbie also references the combined application of UVA radiation and a peroxide solution for increasing the germicide effect.

In the journal “Photochemistry and Photobiology”, September/October 2004, an article: “Photodynamic Treatment of the Dermatophyte Trichophyton rubrum and its Microconidia with Porphyrin Photosensitizers” of Smijs et al. is published. In this article, the treatment of nail fungus with the aid of the photodynamic therapy (PDT) is recommended, wherein the agent “Sylsens B” is claimed to be particularly favorable for killing nail fungus. Red light is the preferred light radiation because it penetrates deeper into the tissue than violet or blue radiation in which the absorption of porphyrines is much higher but the depth of penetration of the radiation into the tissue is too small.

In the treatment of nail fungus which proves to be extremely resistant to all conservative treatment methods, it is important to reach also the visually obscured area underneath the nail plate and to efficiently combat the fungus at that location because it would else spread again after a certain amount of time.

### Specification of the Invention

An object of the present invention is to provide a device for the effective optical treatment of nail fungus diseases which is simple and safe to operate. This object is solved by the treatment device defined in claim 1. The dependent claims refer to preferred embodiments.

The present invention represents an optical irradiation device for the treatment of nail fungus suitable for the practice which corresponds to the following requirements:

A positive treatment effect has to be detectable already within the first session at the dermatologist or podologist. The substantially complete removal of the nail fungus visible from outside needs to be obtainable after a few (3-5) sessions in the practice.

It has to be possible to simultaneously irradiate all 5 toenails in an irradiation time of about 10 minutes and to thereby already obtain a first positive effect. In order to obtain this effect, the optically effective beam power density on the nail surfaces has to be very high, i.e. in the range of >50 mW/cm².

In combination with the optical irradiation, externally applied ointments, gels, pastes or liquids with a peroxide content can cause a softening of fungus-infected nail material which can then easily be removed mechanically.

The device has to be constructed in such an easy to handle way that the podologist can conveniently treat one foot and prepare it for the optical irradiation while the second foot is irradiated at the same time.

The dermatological irradiation device can optionally have different optical spectral ranges:

For the combined method of an irradiation and simultaneous application of a peroxide containing substance onto the one or several affected nails either UVA radiation alone (320 nm<λ<400 nm) or UVA+blue radiation (320 nm<λ<500 nm) or only short-wavelength visible radiation without UV components, e.g. in the wavelength range 380 nm<λ<500 nm or 390 nm<λ<450 nm, is recommended.

The radiation in the violet spectral range can also allow a very favorable fungus fluorescence diagnosis. The spectral requirements for the application of the dermatological irradiation device according to the invention for the irradiation of limited inflammation areas in the case of neurodermatitis, psoriasis or acne can be covered by the spectral ranges required for the nail fungus as well.

In the case of using a conventional gas discharge lamp, the dermatological irradiation device according to the invention comprises four units:

a) A radiation source comprising a housing and a gas discharge lamp with an elliptoid reflector forming a beam focus. The spectral range can be varied by a filter wheel having different band pass filters which is arranged in the beam path between the reflector opening and the focus. A shutter, an intensity control and a timer which controls the shutter are also components of the radiation source.

b) A flexible light guide, preferably a liquid light guide which guides the desired radiation out of the housing and whose light entrance end is arranged in the focus of the reflector lamp. The light exit end of the liquid light guide opens into the applicator part which is configured according to the respective medical application.

c) A beam cross-section converter receiving the radiation of the liquid light guide and converting the circular light ray to an elongated rectangular beam profile which covers the foot nail ledge well. The cross-section converter is constructed according to the principle of the shoe mark is detector of the German patent application DE102005022305.

In principle, the cross-section converter can also be constructed from a triangular thick glass plate having polished surfaces or from a combination of crossed cylindrical lenses of silica glass or from a combination of cylindrical lenses and spherical lenses or simply from a dispersing lens.

d) An applicator part. In the case of the nail fungus irradiation with the possibility of a simultaneous irradiation of all five toenails or fingernails, the applicator part includes a shield housing substantially surrounding the foot or the hand of the patient. A base plate on which the foot or the hand is positioned and which adds stability to the applicator part.

In the applications for irradiating limited inflammation areas as in the case of neurodermatitis or psoriasis, for example, the applicator part and the cross-section converter can either be omitted completely, i.e. the unmodified light exit cone of the liquid light guide, possibly in connection with the beam homogenizer from the German application DE102009021575.1 is simply used or a collimator optic or a focusing optic added to the light exit sleeve of the liquid light guide is used as the applicator part.

If a rectangular or square beam profile is required, a beam cross-section converter added to the light exit of the liquid light guide is used in a way analogous to the toenail irradiation.

In an alternative technical variation, the toenail or fingernail irradiation device includes light emitting diodes instead of a gas discharge lamp and doesn't use a light guide.

### SHORT DESCRIPTION OF THE DRAWINGS

The dermatological irradiation device according to the invention is described in further detail below with respect to FIGS. 1 to 11.

FIG. 1 is a perspective view of the optical irradiation device of a dermatological treatment device according to a first embodiment of the invention,

FIG. 2 is a perspective view of a portion of the irradiation device according to the first embodiment,

FIG. 3 is an exploded view of a portion of the irradiation device according to the first embodiment,

FIG. 4a is a top view of a portion of the irradiation device according to the first embodiment in a first rotational position,

FIG. 4b is a top view of a portion of the irradiation device according to the first embodiment in a second rotational position,

FIG. 5 is an exploded view of the optical irradiation device of a dermatological irradiation device according to a second embodiment of the invention,

FIG. 6a is a bottom view of the irradiation device according to the second embodiment,

FIG. 6b is a perspective view of the irradiation device according to the second embodiment,

FIG. 7 is a cross-section side view of the irradiation device according to the second embodiment,

FIG. 8 is a cross-section side view of the optical irradiation device of a dermatological irradiation device according to a third embodiment of the invention,

FIG. 9 is an exploded view of the irradiation device according to the third embodiment,

FIG. 10a is a bottom view of the optical irradiation device of a dermatological irradiation device according to a fourth embodiment of the invention,

FIG. 10b is a detailed view of the illumination source of the irradiation device according to the fourth embodiment,

FIG. 10c is a cross-section side view of a portion of the irradiation device according to the fourth embodiment,

FIG. 11a is a side view, partially as a cross-section, of the optical irradiation device of a dermatological irradiation device according to a fifth embodiment of the invention, and

FIG. 11b is a bottom view of the irradiation device according to the fifth embodiment of the invention.

### DETAILED DESCRIPTION OF SEVERAL EMBODIMENTS

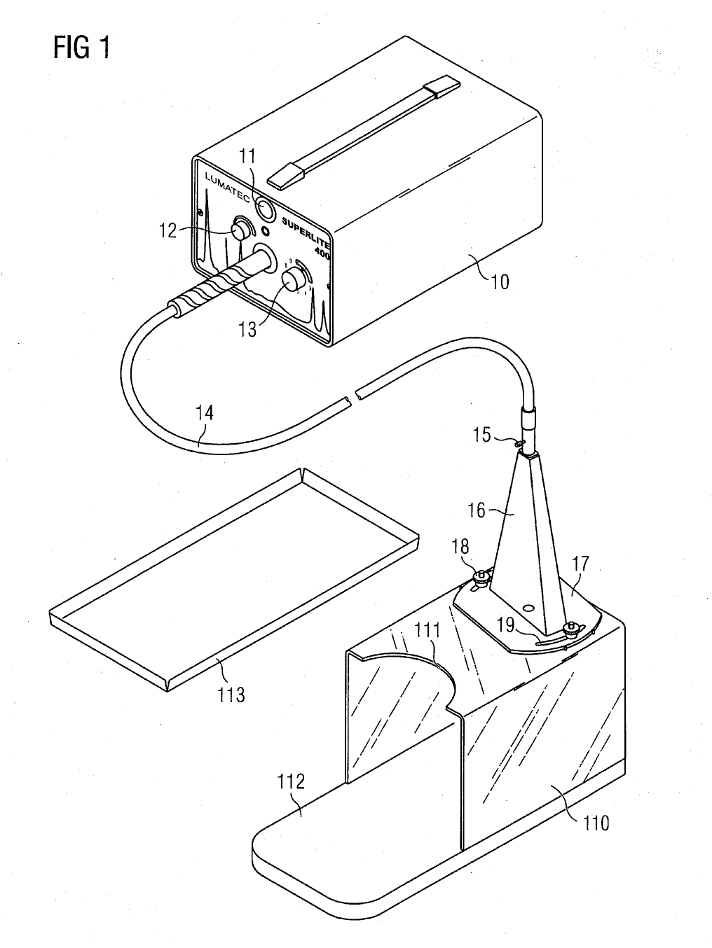

FIG. 1 shows the overall arrangement of a toenail irradiation unit. The lamp housing (10) includes the optical emitter, preferentially an ultra-high pressure mercury lamp having an Hg vapor operating pressure in the range of 100-200 bar, an electrode distance of about 1-2 mm and an electrical input power between 50 watt and 350 watt. The lamp bulb is cemented into an elliptoid reflector generating a beam focus having a light-active cross-section () of about 3-10 mm. The elliptoid reflector is coated with a dielectric multilayer thin-film coating so that a high reflectivity over a broad bandwidth in the whole spectral range of about 280 nm to 1000 nm is guaranteed.

Preferred lamps are the reflector lamp HXP R120W45C VIS or UV of the company Osram® with an electric power of 120 watt or 200 watt or an UHP (Ultra High Pressure) lamp of the company Philips® in a similar electrical power range, for example. The elliptoid reflector can also be a UV reflector with a specially high reflectivity in the UVB, UVA and blue range of the spectrum, wherein the reflector in the green-yellow-red spectral range has a lower reflectivity. Other lamp types are conceivable as radiation sources such as high-pressure Xe lamps, even pulsed Xe lamps or high-pressure or medium-pressure Hg lamps or tungsten halogen lamps or one or several LED arrays emitting in the UVA range at 365 nm or in the violet spectral range around 405 nm, for example.

The interior of the lamp housing (10) includes a filter wheel which can be operated by the exterior rotating knob (13). Thereby up to twelve different spectral ranges can be selected, out of which the following are important for the applications in question here:

1) Full white spectrum without UV, 400 nm-1000 nm; application: nail fungus, also in combination with peroxide gels or pastes, a fungicide agent containing ointments, pastes or oils. Output power of the liquid light guide (output): about 10 watt.

2) UVA+blue: 320 nm-500 nm; application: nail fungus, in particular in combination with a peroxide containing gel or a peroxide containing paste or a peroxide containing liquid and/or porphyrine. Output power of the liquid light guide (output): about 8 watt.

3) Blue without UV: 400 nm-500 nm; application: nail fungus, also in combination with peroxide and/or porphyrine or neurodermatitis, psoriasis. Output power of the liquid light guide (output): about 5-10 watt.

4) UVA: 320 nm-400 nm; application: irradiation of nail fungus in combination with a peroxide containing gel or a peroxide containing paste or a peroxide containing liquid. Output power of the liquid light guide (output): about 2-4 watt.

5) Violet: 380 nm-430 nm; application: fluorescent observation of nail and skin fungus, irradiation of nail fungus in combination with peroxide, psoriasis, neurodermatitis, acne. Output power of the liquid light guide (output): about 2-4 watt.

The output powers measured in watt relate to a liquid light guide series 300, 5 mm×1200 mm of the company Lumatec®.

The lamp which is used here is a 120 watt HXP reflector lamp of the company Osram® or a HXP lamp having a power of 200-300 watt. The radiation of the lamp is coupled into the light entrance end of the flexible liquid light guide (14) in the housing (10) and guided into the light entrance opening of the cross-section converter (16) which is perpendicularly positioned here, and it is fixed by means of the set screw (15) at this position, wherein the rotatability of the liquid light guide in the housing of the cross-section converter (16) is to be maintained, however. The liquid light guide used here has been described in the German patent DE4233087, for example. As the liquid core of the liquid light guide, the solution CaCl₂ in H₂O and, because of its better transmission for red light, also the deuterated variant, namely CaCl₂ in D₂O, can be used Typical dimensions of the liquid light guide are: length 100 cm-200 cm, diameter of the light-active core about 3 mm-8 mm, preferably 5 mm or 5-6.5 mm.

The lamp housing (10) includes other operating elements for the intensity regulation (12) and for the light shutter (11). A timer limiting the exposure time by controlling the shutter can also be integrated into the lamp housing (10).

The cross-section converter (16) is mounted on a rotating table (17) which is attached to the top of the housing (110) by means of 2 knurled screws here. The housing (110) serves for receiving the foot or the hand of the patient in order to irradiate all five toenails or fingernails with light. The housing (110) has the primary function of screening light because otherwise neither the patient nor the physician or the podologist could bear the extreme brightness of the scattered light during the light exposition.

Wearing protective glasses as an alternative would be cumbersome. The foot rests on the base plate (112) connected to the housing (110). The housing (110) and the base plate (112) form a kind of shoe (“light shoe”) into which the foot can be pushed. The bulge (111) facilitates the “slipping” into the shoe.

For hygienic reasons, an insert (113) which can be exchanged for each patient, so to say as a disposable part or as a metal plate which can easily be removed and disinfected in liquid media, can be positioned on the base plate (112). The insert (113) can in the simplest configuration be a sheet of paper with upright edges adapted to the light shoe on which the foot of the patient rests during the irradiation. The patient can sit during the irradiation which typically takes about 10 minutes while the podologist treats the second foot (cutting, grinding, preparation for irradiation or post-processing after irradiation).

Because of the high radiation density of the radiation applied to the nail plate (e.g. 50-100 mW/cm²), the application of a ventilator not shown here or at least of shaded ventilation slots at the face of the housing (110) is recommended because of the noticeable heat action so that a maximum irradiation density can be maintained without noticeable pain for the patient. Apart from the glare-shield effect, the shield housing (110) has to have two other functions, however: First, it has to have a certain strongly attenuated optical transparency because this allows the patient or the podologist to observe or control the position of the toenails during the optical irradiation at any time. Moreover, the walls of the housing (110) should satisfy the function of a long-pass filter, i.e. they should be transparent for long-wavelength orange and/or red light and opaque for short-wavelength (blue, green) visible light.

This characteristic is favorable because in the case of an irradiation of the nails with ultraviolet, violet or blue light, most preferably in the spectral range 390 nm<λ<430 nm, the remaining nail fungus can be excited to fluoresce in the orange, reddish spectral range which is very favorable for the diagnosis of the nail fungus. This additional optical characteristic of the housing (110) implies the important advantage for the patient and the podologist that also the status of the fungus infestation can be evaluated at any time by the dermatological irradiation device according to the invention, namely much more clearly than it would be possible in the case of an irradiation with strong white light.

One configuration of the housing (110) which has proved favorable for all 3 functions (glare shield, position control, fluorescence diagnosis) is that the walls of the housing (110), i.e. the side faces, the front face, the top surface and the rotating plate (17), are made of 3 mm thick plexiglass which is colored light red or dark red or orange.

The optical transmission (T) of this colored 3 mm thick plexiglass plate can have the following approximate transmission characteristic:

T≈0% in the range of 300 nm<λ<500 nm
T=50% lies in a range of 500 nm<λ<600 nm
T=90% lies in a range of 600 nm<λ<750 nm

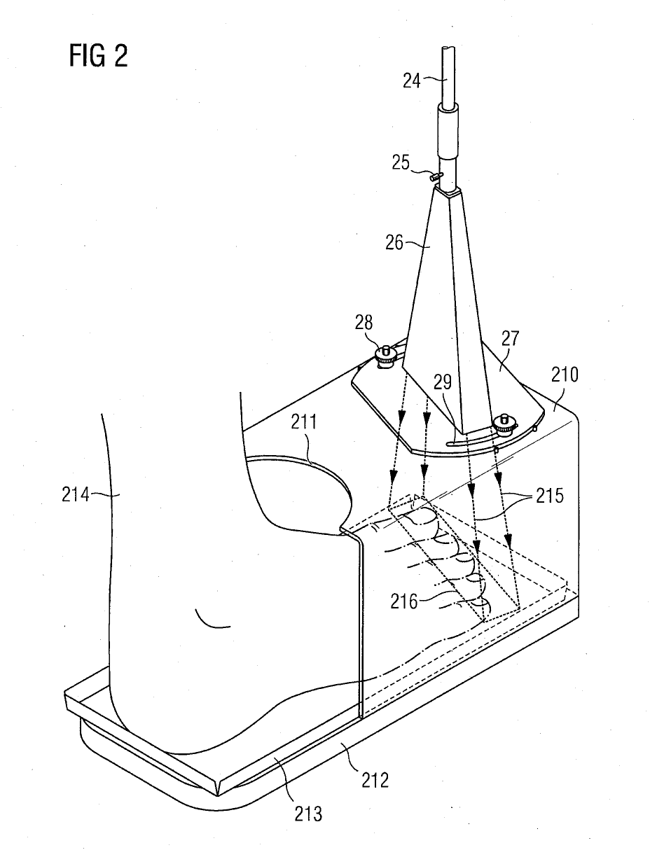

FIG. 2 shows the “light shoe” with the patient foot (214) positioned on the disposable paper padding (213) which is put onto the base plate (212) in advance. The cross-section converter (26) with the liquid light guide (24) in its tapered light entrance end generates a rectangular elongated beam profile (216) indicated here by the geometrical limitation rays (215). The beam profile (216) completely overlaps all five nail plates of the toes, wherein a certain overlap of about 10 mm at each of the four limitation lines is allowed or necessary for guaranteeing a complete beam coverage of the toenail ledge under consideration of different foot sizes and individual anatomic differences.

A further increase of the area of the light profile (216) is not recommended because otherwise an unnecessary reduction of the optical power density measured in W/cm² on the area of the toenail to be irradiated would be the consequence. In general, the optical power density should be as high as possible in the practice operation, i.e. it should be close to the limit at which thermal pain would be generated because else no sufficient therapeutical effect could be obtained in a reasonable time tolerable for the practice operation in the order of 5-20 minutes.

An example for the practice treatment of nail fungus will be described in a short form here for the sake of a better understanding.

A therapeutical effect after an irradiation in the practice is only obtained if the fungus-infected nail plate to be irradiated is covered by a peroxide containing gel (paste) or wetted with a peroxide containing liquid before the irradiation. An effective peroxide containing gel or an effective peroxide containing paste which is preferable over a liquid can comprise the following components, for example:

Gel+carbamide peroxide, wherein the gel can consist of the gel former PCN 400 (chemical name: sodium carbomere or sodium polyacrylate), mixed in H₂O, and the peroxide is mixed in the form of the carbamide peroxide in equal weight amounts with the PCN 400 gel. The overall peroxide content is then approximately 15% (weight ratio). The addition of carbamide peroxide can also be selected substantially higher such as 150 or 200 percent by weight in relation to the weight of the gel or more specifically the gel amount.

Organic peroxides such as t-butyl hydroperoxide, Di-t-butyl peroxide, peroxyacetic acid or dibenzoyl peroxide can also be used for the substance to be applied which have a lower content of “active oxygen” than hydrogen peroxide, however. The organic peroxides can be represented by the structural formula R—O—O—R, wherein the residues R are equal or unequal and can be H-, alkyl-, aralkyl-, acyl- or aroyl-. Especially preferred examples for these are t-butyl hydro-peroxide or peroxyacetic acid.

Another particularly effective paste having a high content of hydrogen peroxide can consist of the following components:

A: aqueous solution of hydrogen peroxide
B: glass powder
C: one or more alkalines or NH₄ or heavy metals or pure carbon
D: one or more carriers
E: one or more tensides
F: photochemical sensitizers

The component A consists of an aqueous 30 to 40% hydrogen peroxide solution, for example, which is commercially readily available. Of course, this solution can also be diluted with H₂O if a reduced effect is desired, e.g. to an H₂O₂ content of only 15-20% or even less.

The component B can consist of an SiO₂ powder, wherein the sizes of the glass particles can vary from only 10 nm to 0.1 mm. The glass from which the powder is made doesn't necessarily have to be pure silica glass. It can also comprise other additives common in the preparation of glasses such as alkaline or earth alkaline oxides or boron (such as the “glass bubble” powder from 3M® from which the hollow glass beads are made). A pure highly dispersive SiO₂ powder with a particle size in the nanometer region around 12 nm has proved effective for the component B. The so-called “Aerosil” SiO₂ powder with particle sizes in the nanometer region can also be used. Adding only a few percent by weight Aerosil or highly dispersive SiO₂ powder (approximately 10% by weight) to an aqueous 30% hydrogen peroxide solution and mixing this solution yields a pasty substance suitable for applying to the nail plate which is still sufficiently transparent for short-wavelength light.

The optical transparency of the paste is important because it is exactly the generation of oxygen or the formation of OH radicals at the interface between the nail plate and the paste which matters. The small solid amount (SiO₂) in this paste enables that the high concentration of H₂O₂ of originally 30% lessens only slightly, benefitting the desired effect, namely the softening of the fungus-infested nail substance in only a few minutes. This softening effect is caused by the highly reactive oxygen or the strong oxidation effect of the OH radicals generated by the radiation enhanced by the hydrogen peroxide excited by the light irradiation.

The pasty consistency of the mixture of the components A and B in contrast to a liquid consistency, for example in the case of using the component A alone, enables a better differentiation between the etching hydrogen peroxide and the skin tissue adjacent to the nail plate which can then be well protected by fat containing ointments against the application of the peroxide containing paste.

The component C includes catalysts for an acceleration of the decay of the hydrogen peroxide. Those can be lyes such as KOH or NaOH. NH₄ (ammonium) or small additions of heavy metals or a small amount of finely grained carbon also favor this decay. The amount of the substances of the component C lies in the range of a few percent by weight and can even be below 1 percent by weight in relation to the overall weight of the paste. The substances of the component C can also be left out altogether because the utilization period or the storage time of the paste mixed by the components A and B strongly diminish and because on the other hand the high-power irradiation of the paste with short-wavelength light in the range of 320 to 500 nm by the irradiation device according to the invention generates sufficient oxygen or OH radicals for the desired effect of the softening of the fungus infected nail material. On the other hand, if a longer treatment time is accepted, the irradiation can be dispensed with altogether if the addition of substances from the component C is properly adjusted. However, the softening effect is considerably better if light is added.

The component D in the composition of the paste describes the carrier substances known in medicine and cosmetics. As examples for these, dimethyl sulfoxide or dimethyl sulfone can be named. These substances increase the depth of penetration of the paste into the tissue. These carrier substances can be used individually or in combination in the composition of the paste, however, the effect of the paste, in particular in combination with the intense light, is usually sufficient even without the carrier so that the carrier substances can usually be left out.

The admixture of a small amount of a tenside (component E) improves the wettability of the nail plate by the liquid phase A. Examples of anionic tensides that can be used are linear alkylbenzene sulfonates, an example of a cationic tenside that can be used is cetyl trimethyl ammonium bromide, and an example of a non-ionic tenside that can be used is polyalkylene glycol ether.

The addition of photochemical sensitizers (component F), i.e. substances which are able to transfer light energy to the peroxide, can make the use of UV light unnecessary or increase the effect of longer-wavelength light. Suitable examples are dyes such as Eosin Y (tetrabromofluorescein), Erythrosin (tetraiodinefluorescein), Rose Bengal (tetra-iodinedichlorofluorescein) but also chlorophylles and porphyrines.

In general, the nail plate has to be mechanically roughened before a peroxide containing substance (gel, paste or liquid) is applied because otherwise only a much weaker therapeutical effect is obtained. The roughening or grinding of the nail plate is usually effected with a rotating milling tool in the practice. The roughened nail plate to be treated also has to be absolutely fat-free.

Before the peroxide is applied and before the irradiation, the podologist protects the skin tissue adjacent to the nail, for example with a sun lotion having a high sun protection factor against UVA radiation and the slightly etching peroxide gel. However, a fat-containing protective lotion such as Vaseline™ or Bepanthen™, if applied to the tissue, is also sufficient.

After the application of the peroxide gel or the peroxide containing paste to the nail plate, the toenails in the light shoe are irradiated for about 10 minutes, in particular with radiation in the spectral range of UVA plus blue, i.e. in the range 320 nm≧λ≧500 nm or even in the UV-free spectral range 380 nm<λ<500 nm. The overall output from the cross-section converter amounts to about 5 watt in this case. The radiation applied to each nail plate also has a power density of approximately 100 mW/cm². The applied radiation dose can thus be up to approximately 60 Joule/cm².

After the irradiation has been performed, the podologist determines that the largest part of the fungus infected nail material has been softened and can be scraped or cut away. The not fungus infected still healthy nail material will stay substantially unchanged. The same irradiation procedure in combination with the peroxide gel can also be repeated immediately.

In this approach, a selective destruction or softening of the fungus infected nail material, here effected by radiation-induced oxidation, is observed, whereas the healthy nail material is obviously insensitive to the highly reactive oxygen or OH radical released from the peroxide by the intense short-wavelength optical radiation. The fungus infected nail material rich in organic substances is oxidated by the released singular oxygen or the generated OH radical. The nail fungus or the organic component in the nail plate thus virtually suffers a cold burn.

Thus, the largest part of the nail fungus can be destroyed in one session or at least softened and mechanically ablated. 2 to 3 further similar treatments can be required after that, depending upon the degree of severity of the original fungus infection. If the same high radiation dose is applied to the fungus-infested nail, however without the use of peroxide, no therapeutic effect at all is gained even after several repetitions of the irradiation and even in the case of a daily irradiation over weeks. The therapeutically important softening effect is obtained exclusively by the action of the peroxide containing substance effected here by the radiation and/or possibly catalyzed by additives from the component C to the paste.

As an alternative, also the application of protoporphyrine IX in an aqueous or alcoholic solution instead of peroxide in combination with the irradiation was tried. Such an aqueous solution with protoporphyrine IX can also be brought to a pasty consistency by admixture of highly dispersive SiO₂ (e.g. 5% by weight), for example, which is advantageous for the application to the nail plate. Furthermore, one or several carriers (e.g. DMSO or dimethyl sulfone) can also be admixed to such a porphyrine solution or paste in order to increase the depth of penetration of the porphyrine into the tissue. The light wavelength would then conveniently be centered around 405 nm or lie in the red spectral range, e.g. at 630 nm±10 nm.

With this variant which originates from the practice of the photodynamic therapy (PDT), a strong oxidation effect with a destructive action against the nail fungus or a softening of the infected nail material could be observed as well. However, the disadvantage of this method is that the nail is darkened by the porphyrine. The color change remains after the irradiation so that the patient has the optical impression that the nail fungus which changes the color of the nail as well did not improve. The cosmetic effect thus leaves a lot to be desired in this application variant although the color modification caused by the porphyrine can largely be bleached out by a subsequent treatment with peroxide as described above.

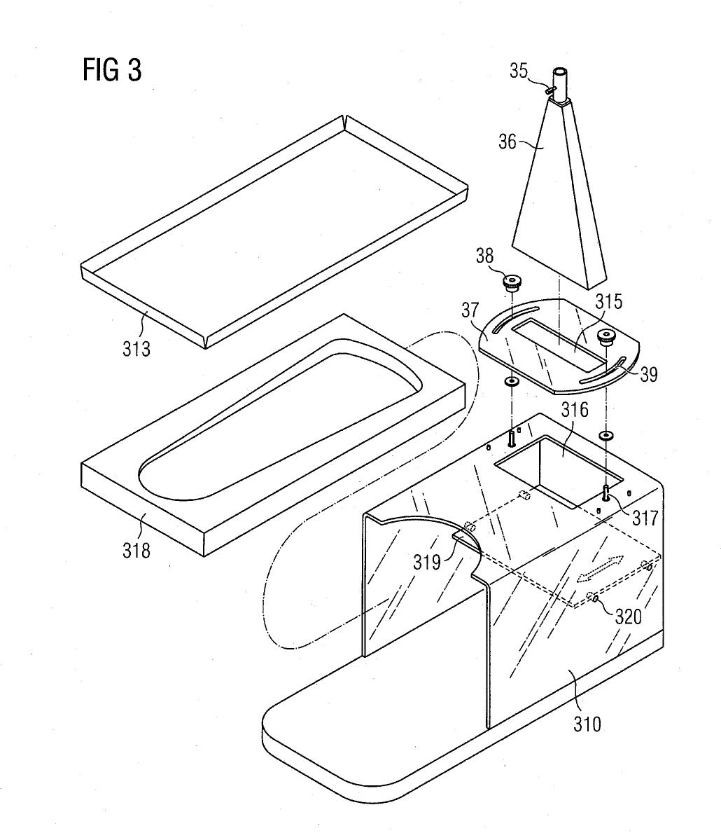

FIG. 3 again shows the construction of the “light shoe” in detail. Instead of the simple paper foot padding (313), a foot padding (318) having a foot bed is illustrated here as well. This padding has the advantage that the foot is positioned more accurately in the light beam. It can also be formed as a disposable part, e.g. if it is made of styrofoam. Here, the shield housing (310) has a rectangular recess (316) not recognizable in FIG. 2 which can be covered by a transparent (also in the UVA range) plexiglass plate glued to the inside of the housing. This recess is required because of the necessary rotatability of the cross-section converter by up to ±30° around its vertical axis relative to the transverse axis of the light shoe, wherein no optical shading of the radiation emitted by the cross-section converter may occur.

FIG. 2 shows the most extreme rotational and angular position of the cross-section converter as an example which is required for the irradiation of the right foot, in particular because the toenail ledge approximately forms an angle of 20° -30° with respect to the transverse axis. If the cross-section converter would not be rotatable, the rectangular beam profile (216) in FIG. 2 would have to be broader with a corresponding decrease of the available beam power density (mW/cm²) at the nail plates. If the left foot is to be irradiated in FIG. 2, the knurled nuts (28) in FIG. 2 or (38) in FIG. 3 are slightly released, and the cross-section converter (26) or (36) is rotated to the alternative position from +30° to −30° . The stop of both rotational positions is defined by the two circular grooves (39) in the rotating plate (37) in FIG. 3 and the screw pins (317) protruding into it. The rotational position is fixed by the knurled nuts (38). For anatomically deviating feet, any intermediate rotational position between +30° and −30° can be set as well. The rotating plate (37) has a rectangular recess (315) matching to the light exit opening of the cross-section converter (36). The rotating plate (37) and the cross-section converter (36) are fixedly glued to each other or mechanically connected to each other by other means.

FIG. 3 also shows an insertion plate (319) which can optionally be inserted into the interior of the shield housing (310) resting on four pins (320), namely at a height of approximately one third above the bottom plate. This plate should be as close as possible to the toenails. It consists of plexiglass which is colored with a very high concentration with one of the dyes of a Lumogen® series. Those can be the dyes Lumogen® red or Lumogen® orange or Lumogen® yellow, for example, which are all dyes of the chemical group of perylene dyes. In the case of coloring with Lumogen® red, this plate fluoresces in red light with an emission between 600 nm and 700 nm, the maximum of the emission lying around 630 nm. Instead of a fluorescent plane plate, a U-shaped arched plate made of plexiglass which is doped with the fluorescence dye and which can more rapidly be inserted into the light shoe can be used as well.

In the case of an optical excitation in the short-wave-length UVA-blue-green-orange range, a red or orange emission radiation centered around 630 nm can thus be generated by fluorescence and used for irradiating the nails.

A radiation centered around 630 nm is favorable for the treatment of nail fungus because first it penetrates much deeper into the nail and the surrounding tissue than short-wavelength UVA and blue radiation and because the porphyrines produced by the body and externally applied porphyrines have a side lobe of the absorption at 629 nm which can be important for combating the nail fungus infection by the method of photodynamic therapy. This plate (319) doped with a dye fluorescing in the red region thus has the function of a wavelength shifter. By the accidental peak emission at 630 nm, this fluorescence plate doped with Lumogen® red or Lumogen® orange can also be useful in all other applications in which porphyrines produced by the body or externally applied porphyrines play a role for the healing such as in the light treatment of neurodermatitis, psoriasis, acne or other skin diseases.

In addition to the irradiation with light which penetrates deeper into the tissue, an ointment or an oil or a lacquer with a fungicide agent can be applied in which case the active substance can be better absorbed by the tissue. However, this method is only successful in the case of a long-term application. It is indicated for the post-treatment of the light-peroxide-method in order to destroy even the last remnants of the remaining fungus spores.

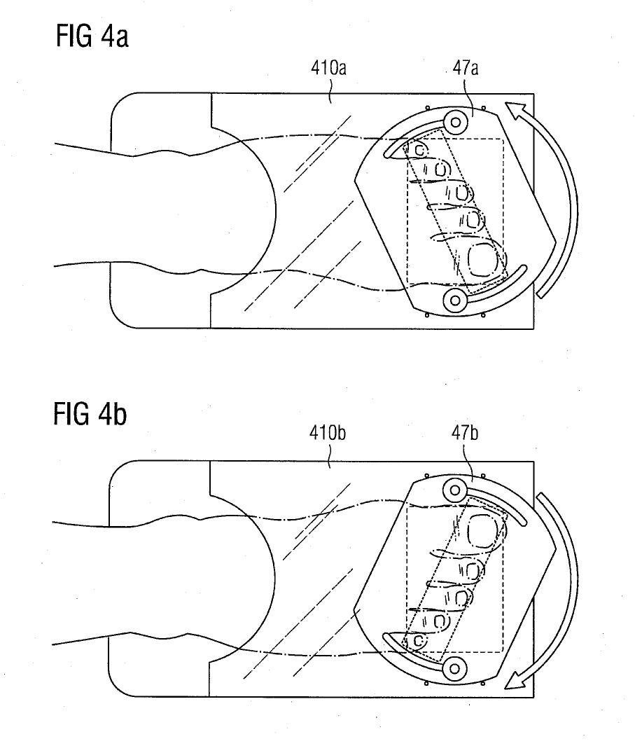

FIGS. 4a and 4b again show the positions of the cross-section converter including the rotation plate (47a) and (47b), respectively, in the plan view of the light shoe in the case of irradiating the toenails of the left foot under the shield housing (410a) and the right foot under the shield housing (410b) transparent for red light.

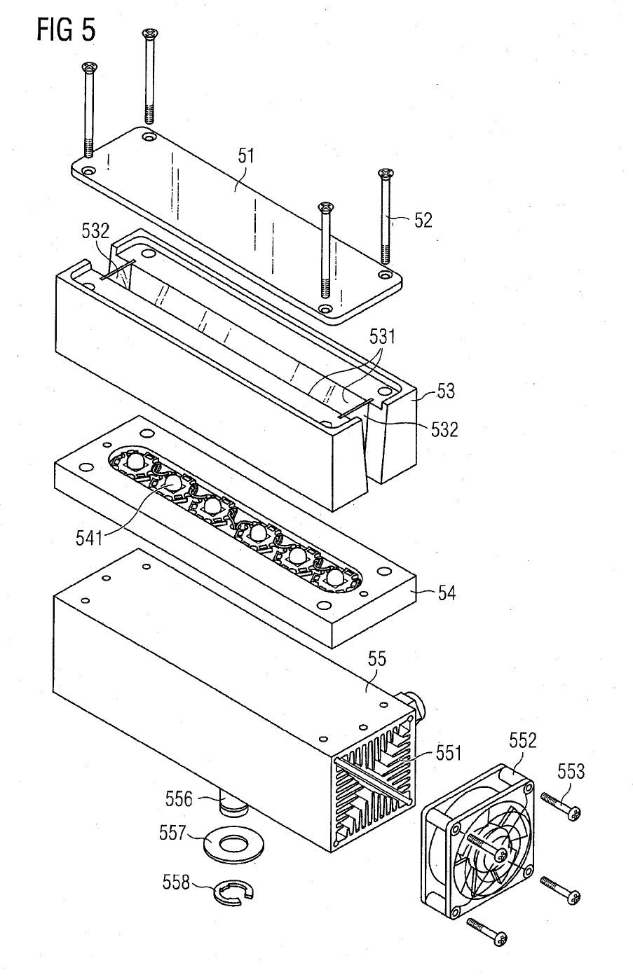

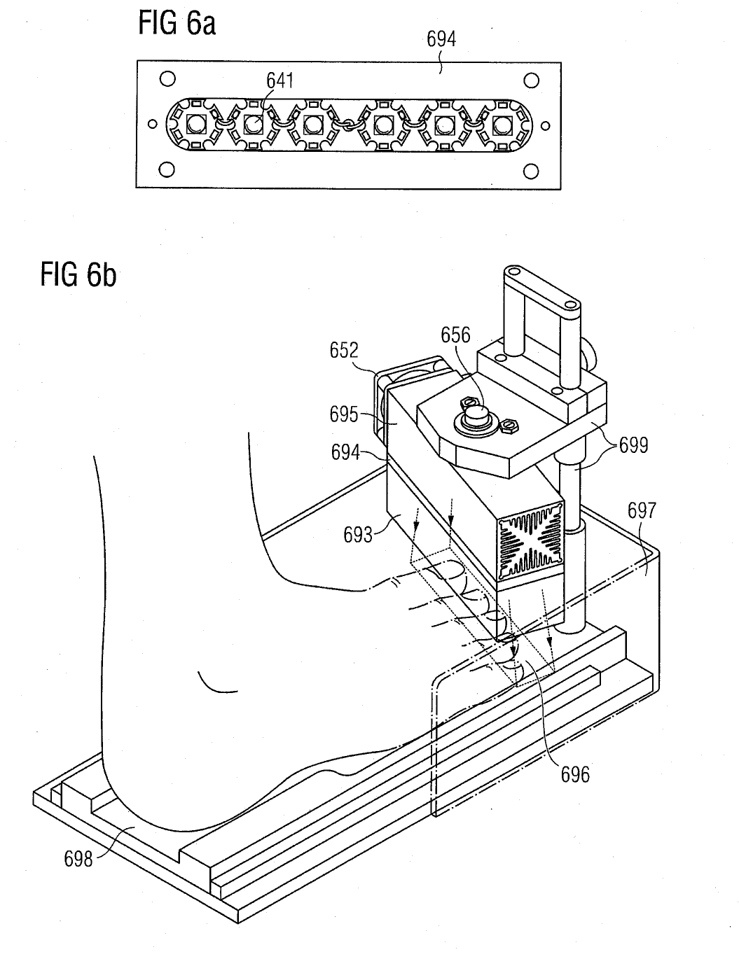

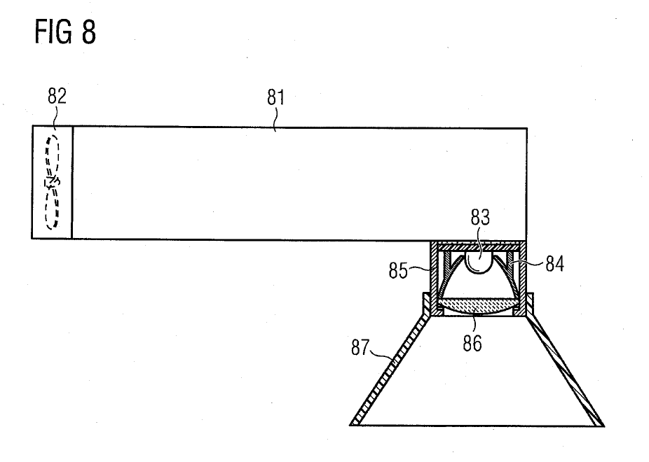

An alternative toe or fingernail irradiation device is described with respect to FIGS. 5-11 which also generates an intense light stripe covering the nail ledge by means of linearly arranged high-power light-emitting diodes (ref. FIGS. 5-6) or rather circular light spots as in FIGS. 8-11.

FIG. 5 shows the structure of such an irradiation device. Six high-power LEDs (541) are glued with a good heat contact, e.g. by means of a heat conductive adhesive, onto an approximately 10 mm thick copper plate (54). The electrical power of such an LED is about 10 watt in this example, comprising an array of four series-connected lower-power individual diodes. Each three of these LED arrays are connected in series, and the two groups of three arrays are connected in parallel. The maximum overall electric power is about 60-90 watt, the maximum voltage connected to each three series-connected diodes is about 30-60 volt, and the current flowing through the diodes is 500-1500 mA. The electric voltage applied to the irradiation device is kept in the low-voltage range in this way, which is important for the safety of the patient.

The plate (54) including the diodes is connected in a heat-conductive manner to a heat radiator (55), e.g. made of aluminum, which can have cooling fins (551) directed to the inside. The stud (556) attached to the heat radiator (55) as well as the washer (557) and the snap ring (558) serve for supporting the irradiation body as illustrated in FIG. 6b. The reflector (53) is fastened by four screws (52) at the surface of the copper plate (54) facing the diodes, wherein the reflector is also sealed and protected by means of the four screws (52) by the light exit window (51) which consists here of UV-transparent plexiglass, for example.

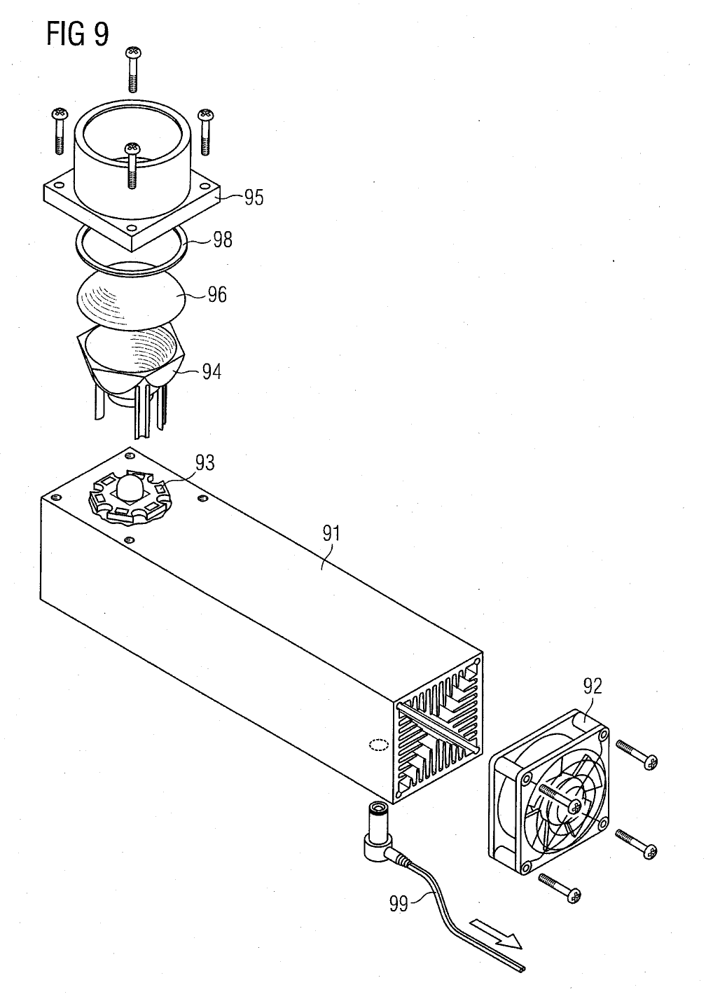

The reflector (53) has two V-shaped reflector plates (531 and 532) externally opening towards the light exit side which can consist of highly reflective aluminum with an SiO₂ protective layer. Instead of a single V-shaped reflector (531, 532), each of the LED diodes (or arrays) can be equipped with a round reflector similar to the one illustrated in FIG. 9 (94). It is also possible that a convex attachment lens is provided on each individual reflector (as illustrated in FIG. 9 with reference number 96), or an elongated cylindrical lens can be provided which covers all reflectors. The fan (552) provides air throughput through the radiator (55) and is attached by four screws (553) at the same.

FIG. 6a again shows the arrangement of the 6 high-power light emitting diodes (641) on the copper substrate (694) in further detail. The arrangement of the diodes is substantially linear but not necessarily equidistant. The distance of the two central diodes can be the largest, and the distance of each two adjacent diodes from the center to the edges towards the left and the right side decreases. In this manner, the light stripe (696) lying over the toenails receives a sufficiently homogenous beam power density. In this example, 6×10 watt LED Engine® LEDs having a peak emission at about 400 nm±10 nm were chosen.

FIG. 6b shows the entire mounted irradiation complex comprising the fan (652), the radiator (695), the copper plate (694) and the reflector (693) supported by the stud (656) at a supporting frame (699). The entire irradiation body can be rotated by about ±30° along the axis of the stud (656) so that the light stripe (696) can be aligned for the left and the right foot in analogy to the irradiation device of FIG. 2. Also in this case, the light radiation should in principle be limited to the ledge of the 5 toenails in order to maximize and utilize the beam power density generated by the six expensive high-power diodes.

In the above example with the six linearly arranged light emitting diodes having an electric power of 10W and an emission in the violet spectral range at 390 nm<λ<410 nm, a beam power density of about 10-300 mW/cm² could be measured at the nail plates of all five toes in a distance of about 3-4 cm between the nail plates and the reflector (693). This power density is sufficient for softening a fungus infected nail plate area if a highly-concentrated hydrogen peroxide containing paste (or solution or gel) is applied simultaneously as described above within an irradiation time of only 10 minutes. Further, a considerable fluorescence of the present nail fungus can be observed at the wavelength of about 400 nm, if necessary even without using a long-pass filter, even if a long-pass filter having a transmission in the yellow-orange range and an absorption in the blue-green range improves the contrast and spares the eyes of the observer.

In analogy to the irradiation device having a light guide and a cross-section converter, the foot of the patient rests at a padding (698) having a foot bed. A cover (697) of the padding (698) for optical shielding purposes can also be provided, and it can consist of yellow or orange colored transparent plexiglass for the above mentioned reasons.

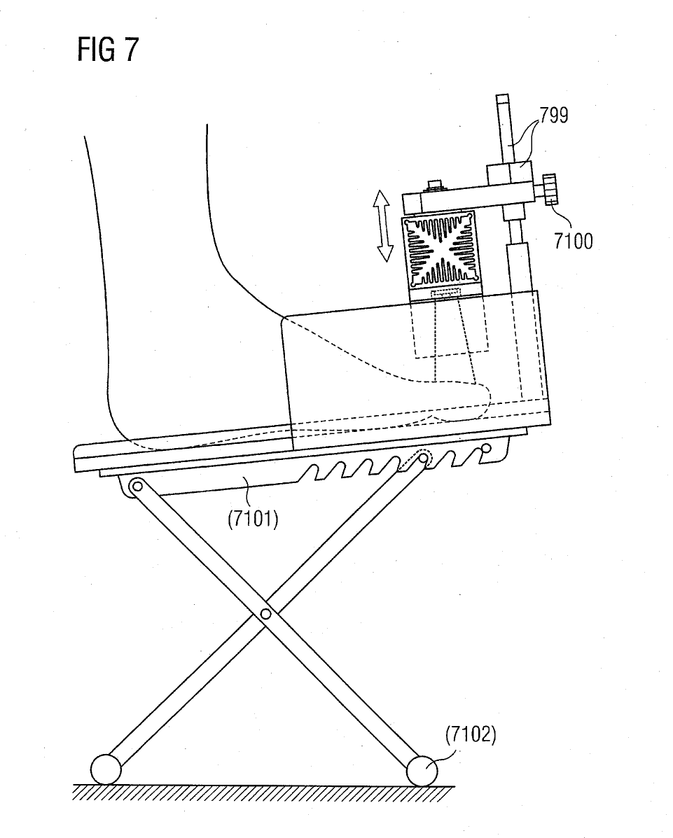

FIG. 7 shows a side view of the LED irradiation unit supported at a supporting frame (799). A height adjustment is possible by the set screw (7100) which is favorable for the fungus-fluorescence diagnosis and for the adjustment of the irradiation intensity. FIG. 7 also includes a support (7101) which is very useful for the operation in the practice of the podologist and whose inclination and height can be adjusted, such as used by guitar players as a foot support. This foot support which is available on the market at a very favorable price and which is optimally suited for the operation in the practice of the podologist can obviously also be used as a support for the irradiation device of FIGS. 1 and 2. The only modification to be performed is a transverse boom on the front floor support (7102) in order to increase the tilting stability.

In contrast to the gas discharge lamp used in the device of FIG. 1, the light emitting diodes usually emit monochromatic light. In the case of using diodes having an emission at 400 nm-405 nm and a high power as in the above example, favorably no UV radiation is required even if the radiation is as close as possible to the limit of the UV range. Even if the oxygen separation or OH radical formation by the radiation effect onto H₂O₂ is more effective in the UV range of the spectrum, the monochromatic radiation at 400-405 nm as generated by high-power LEDs represents a good compromise, in particular if legal regulations stipulate that the exposition of human tissue by UV radiation is to be avoided which is usually the case.

There are also high-power diodes emitting white light or diodes having a monochromatic emission in other spectral ranges which can be used in the irradiation device of FIG.

6b. For the application of irradiating nail fungus in combination with peroxide and/or porphyrine in question here, the following wavelength regions are suitable which are generated by diodes emitting in these wavelength regions:

350 nm<λ<400 nm for nail fungus in combination with peroxide
390 nm<λ<410 nm for nail fungus in combination with peroxide and/or porphyrine, also for the fluorescence diagnosis
400 nm<λ<500 nm for nail fungus in combination with peroxide
600 nm<λ<1000 nm for nail fungus in combination with porphyrine and/or in combination with conventional ointments, oils and lackers containing an agent
400 nm<λ<1000 nm (white diodes, infrared diodes or red emitting diodes) for nail fungus in combination with peroxide and/or porphyrine or with ointments, oils or lackers containing an agent or simply without using any substances.

In the irradiation device of FIGS. 6a and 6b, not only light emitting diodes of a single color but also a mixture of diodes emitting in different colors can be used for irradiating the toe or fingernails. In this way, light emitting diodes emitting in the UVA or violet range can be complemented by those emitting in the blue range, for example, in order to achieve a better penetration of the peroxide containing paste by the light of the longer wavelength. Optionally, photochemical sensitizers can also be admixed to the paste or substance. Also for reasons of increasing the penetration depth of the radiation, in the case of using porphyrine or a fungicide agent containing ointments or solutions, a mixture of :LEDs emitting at approximately 405 nm and those emitting at approximately 630 nm or diodes only emitting at longer wavelength, e.g. at 740 nm, 850 nm or 940 nm, can be used.

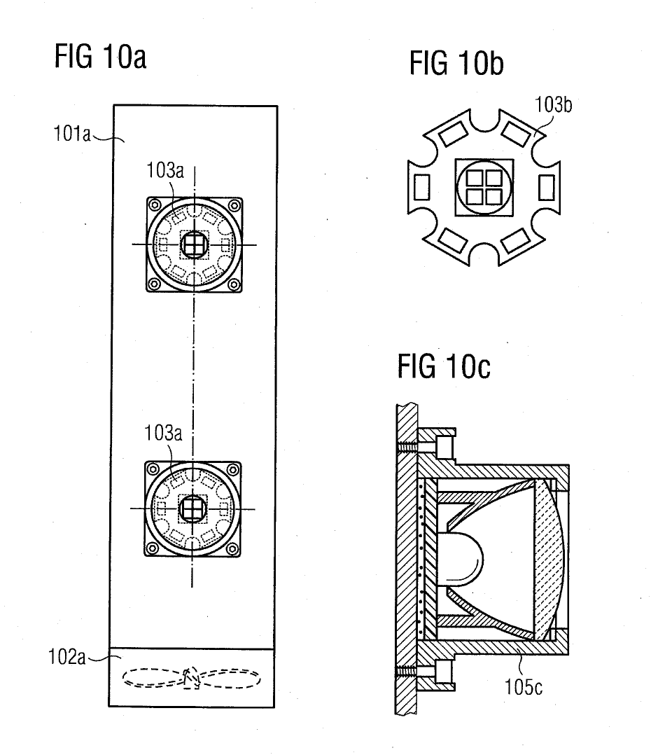

It is equally possible to exchange the LEDs as one complete unit for switching to other wavelengths, i.e. to replace the LEDs of a first emission spectrum by those of another spectrum. The plates (54, 694) with the associated heat radiators (55, 695) in FIGS. 5 and 6 or the heat radiator (101a) with the LEDs (103a) attached to it in FIG. 10a can be easily removably attached to the irradiation device according to the invention, for example, so that they can easily be replaced by replacement elements with LEDs of another emission spectrum.

FIGS. 8 and 9 illustrate a toenail/fingernail irradiation device comprising only one single light diode (83, 93) or only one single diode array (83, 93) consisting of 4 to 6 individual diodes, for example, which can be connected in series or groupwise in parallel. The light emitting diode or the diode array is connected in a highly heat conductive manner to an elongated heat radiator (81, 91) made of aluminum. In this example, the heat radiator (81, 91) has the outer dimensions 30×30×123 mm with cooling fins (551) directed to the inside in analogy to FIG. 5 and a small fan (82, 92) which provides for air throughput in the interior. In this example, the elongated heat radiator (81, 91) can serve as a grip if the irradiation device is to be guided by hand. The radiation of the LED perpendicular or angular to the axis of the hand grip further has a safety effect because the danger of blinding the operator or other persons is less probable by this geometry.

Because of the efficient cooling, LED arrays having an electric power of up to 15 watt can be operated at 100% duty cycle with this arrangement. The emitted radiation of the light emitting diode or the diode array is bundled by a funnel-shaped reflector (84, 94) at the enlarged light exit surface of which a convex lens (86, 96) of silica glass or of normal optical glass or of plexiglass is arranged. In this embodiment, the lens has a focal distance of approximately 3-4 cm and an aperture of approximately 22 mm. The lens (86, 96) and the reflector (84, 94) are framed in the outer lens tube (85, 95), wherein an O-ring (98) of elastic material provides the sealing to the outside.

FIG. 8 also includes a funnel attachable to the lens tube (85, 95) which can carry out up to three functions: a) The optical long-pass filter effect for observing the fluorescence of fungus-infested nail or skin areas. b) A glare shield for the operator and for the patient. The cumbersome wearing of protective glasses can be avoided. c) Maintaining a minimum distance to the irradiated area so that the beam power density at the nail plate or the tissue is not higher than 100 mW/cm², for example.

The LED light source used in this embodiment is an array consisting of 4 individual diodes having a peak emission at λ˜405 nm ±10 nm and an electric input power of 10 to 15 watt. The required electric voltage is about 15 volt (in a series connection of the individual diodes), i.e. it lies in the harmless low-voltage range. The beam output power in the violet range at about 405 nm is at least 2.1 watt. In a distance of approximately 8 cm measured from the lens aperture, a light spot with a diameter of approximately 7 cm is generated so that an irradiation intensity of approximately 50 mW/cm² is available. With this power density in the violet spectral range at approximately 405±10 nm, the desired softening effect of fungus infected nail material can already be obtained within 10-20 minutes. It is a prerequisite that the nail plate is roughened and degreased before the irradiation and that a layer, i.e. a gel, a paste or a liquid including approximately 30 percent by weight H₂O₂ has been applied.

The irradiation device of FIG. 8 or 9 has only one diode array, and not all 5 toenail plates of one foot but only 1-2 or at most 3 adjacent nail plates can be irradiated, which is sufficient in many cases, however.

However, the small dimensions and the low weight (165 g) of this irradiation device allow the irradiation even by hand in the same way as with a torch and not only of toenail fungus but also of fingernail fungus or skin fungus, and they allow to make it visible by fluorescence or generally to irradiate skin tissue by hand. The fact that it is possible to work in the visible spectral range instead of the ultraviolet spectral range and that the device only needs harmless low voltages makes it safe for the patient and also for the operator. Adding 2 or 3 Li-ion battery cells around or on or in the hand grip (81, 91) enables a wireless operation of the device up to one hour increasing the flexibility of its use.

In case of a limitation to the possibility of irradiating only one nail plate, e.g. the nail plate of the big toe, LEDs having a lower electric power such as 5 watt can be used as well. In order to obtain the required power density (−50 mW/cm² and more) for the substantial photochemical effect, simply the distance between the lens and the nail plate is reduced. The electrical power and the irradiation distance can be adjusted so that the optimum power density of the irradiation in the case of a contact between the tissue and the outer edge of the screen (87) exists guaranteeing a 100-percent glare shield and simplifying the treatment procedure. The screen (87) then also serves as a spacer.

Instead of the funnel (87) (or in addition to it), a small tube (not illustrated) in form of a small hat or a cap can be pushed onto the lens tube (85, 95) (e.g. with a clamp fit), the light exit surface of which consists of a transparent plastic material or entirely consists of this material and which can easily be replaced and cleaned. Such a cap allows the direct abutment of the light exit opening to the tissue or the nail plate (or at least its approximation up to the limit of contact) during the irradiation. The paste containing the agent located on the nail plate or the tissue can then obviously be applied to the light exit or abutment surface. The cap should therefore either be designed as a disposable part, or it should at least be easily cleanable.

Suitable materials for the contact with the tissue or for pressing onto the tissue are highly transparent or at least translucent soft silicone® elastomers, highly transparent and soft polyurethane®, PVC®, PE® or other soft, highly transparent plastic materials which adjust to the contour of the nail plate if they are slightly pressed against its surface.

Soft, highly transparent silicone which as a flat press-on body can have a thickness of up to lcm is also preferable because it adapts very well to an outer contour if pressure is applied. The press-on surface or abutment surface can be flat, concave or convex. The agent can virtually be pressed mechanically into the pores of the tissue by the pressure application, and the evaporation and hardening of a gel or a paste or a liquid component during the irradiation can thereby be prevented, and it can thus be obtained a better penetration.

Equally well suited are Teflon® FEP (which still transmits more than 75% at λ=400 nm in the case of a layer thickness of 0.5 mm), Dyneon® THV and generally copolymers or terpolymers of PTFE. The latter carbon fluoropolymers are preferable because of their anti-adhesive property, their chemical inertness and because they can easily be cleaned.

The small tube in the form of a hat or a cap can completely or partially be made of one of the above materials and can preferably be fabricated by the injection-molding technique or lathed from solid material. If the hygienic requirements of the application permit this, instead of the cap, light exit windows can be made of the same materials which are attached directly at the lens tube (85; 95), e.g. by crimping the window at its inner periphery.

The press-on technique is favorable not only in case of using peroxide or porphyrine containing substances with a short-wavelength (λ˜400 nm) irradiation but also in the application of other ointments containing an agent on the skin or on nail plates in the case of an irradiation in the longer-wavelength spectral range (red, infrared). The press-on technique or the maximum approximation of the light exit opening to the tissue further has the advantage that the lowest possible electric power is required for the LED(s), increasing the cost effectiveness.

Instead of an array with an emission around 405 nm, the device of FIGS. 8-9 can also be equipped with an LED array around 365 nm or around 465 nm or for the irradiation of deeper lying fungus spores with diodes emitting at longer wavelengths, e.g. at 630 nm, 740 nm, 850 nm or 940 nm. The LEDs around 465 nm are especially powerful. With the small device according to FIGS. 8-9, a beam output power of almost 3 watt is obtained in case of using an LED array including 4 individual diodes and a total electric power in the blue range at λ≈465 nm of 10-15 watt. The generation of OH radicals by the irradiation of a H₂O₂ containing paste (gel, liquid) with light of this wavelength at about 465 nm is much slower than at λ=405±10 nm. The addition of a very small amount of a catalyst to the H₂O₂ containing paste (gel, liquid) or a photochemical sensitizer also allows this wavelength for the softening of the fungus-infected nail, however with the possible tradeoff of a reduced storage stability of the paste (gel, liquid).

FIG. 10a shows an irradiation arrangement analogous to the one illustrated in FIGS. 8 and 9 which includes 2 (at most 3) LED arrays (103a, 103b) comprising a reflector and a lens, however. The two complete optics setups (again illustrated in a cross-section view in FIG. 10c) are identical to the one illustrated in FIGS. 8 and 9 and are also mounted to an elongated rectangular heat radiator (101a) in a distance of about 40 to 60 mm to each other (in the case of 3 LED arrays in a distance of 20-40 mm). The heat radiator (101a) made of aluminum which has a fan (102a) screwed onto it is slightly larger than the one in FIGS. 8 and 9 in this embodiment. It has the outer dimensions 50×50×120 mm and also has the cooling fins directed to the inside.

The 2 (3) used diode arrays having 4 individual diodes (103b) can emit at 365 nm, 405 nm or 465 nm or in the red or infrared range as well.

The arrangement 10a can simultaneously irradiate all five nail plates of a foot and is conveniently arranged on a light shoe housing analogous to the one illustrated in FIG. (211) instead of the cross-section converter (26) having a rotating plate (27) used there. For this purpose, two (three) pass openings for the two roundish optics setups (105c) in the top plate of the light shoe housing (211) suffice. The rotatability of the irradiation unit of FIG. 10a in analogy to the rotatability of the cross-section converter (26) in FIG. 2 is not absolutely required in this case because the available irradiation area defined by the two (three) slightly overlapping circular light spots in a distance of about 7-8 cm, corresponding to the distance between the lens opening and the nail plate, receives a sufficient beam power density (>50 mW/cm²) from the two (three) LED arrays in order to obtain the desired effect of softening the fungus infested nail material and because it is also sufficiently large for subsequently overlapping the nail plate ledges of both feet.

A rotatability of about ±25° in analogy to the rotatability of the cross-section converter (26) in FIG. 2 of the irradiation arrangement of FIG. 10a is easily possible, however, if instead of the two exterior round holes two angular long holes are milled into the top plate of the light shoe housing (211) of FIG. 2. Of course, the light shoe housing (211) used here in connection with the LEDs not only satisfies the function of a glare shield but also the function of a long pass filter, and it can consist of transparent, orange dyed plexiglass in analogy to the irradiation devices of FIGS. 1 and 2.

The optics setup which collects and bundles the radiation emitted by the LEDs and in general includes the reflector (94) and the lens (96) and which is used in all the irradiation devices described with respect to FIGS. 8-11 can also be slightly modified. It is not required that the reflector (94) has a circular symmetry. It can also have a symmetry of a pyramid base with reflecting interior surfaces, wherein the lens (96) covers the larger light exit surface of the reflector and the LED is positioned in the smaller light entrance opening of the reflector. It is not required that the reflector has a square cross-section. It can also have an elongated, rectangular light exit opening and light entrance opening, in which case it is very similar to the cross-section converter (36) of FIG. 3 but much smaller.

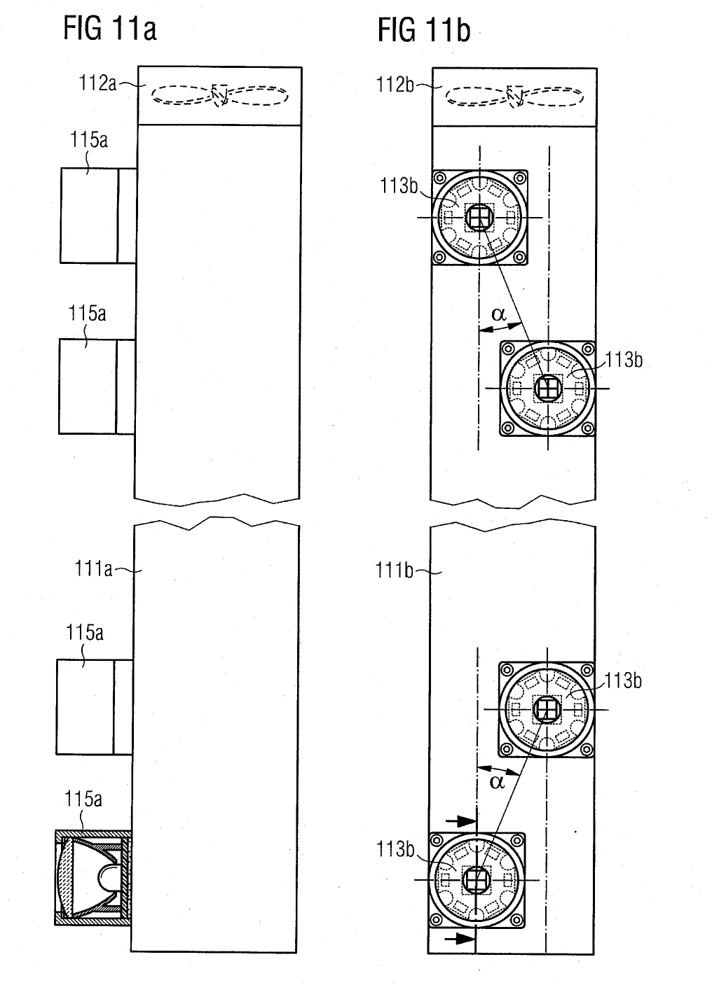

The non-circular symmetry of the reflector (94) can be favorable if 2 light irradiation areas of adjacent LEDs should be merged such as in FIG. 10 or 11 with the smallest possible overlapping area of the light spots in order to obtain the most homogenous possible beam power density in the elongated irradiation field of all five nail plates of a foot. Instead of the spherical lens (96), a transparent plane plate, a cylindrical lens or a diffuser plate can be used as well.

FIG. 11 (11a, 11b) illustrates a device including four LED light sources (113b) for the irradiation of all ten nail plates of both feet. The LEDs (113b) are mounted on the elongated heat radiator (111a, 111b), wherein each two of the outer LEDs are slightly offset with respect to each other, e.g. by an angle a of about 20° in order to take into account the angular position of the nail plate ledges of both feet with respect to each other. The heat radiator (111a, 111b), the fan (112a, 112b) and the optics heads (115a, 115b) are similar or identical to those in FIGS. 10a and 10b. The light shielding housing not illustrated here and the foot padding as illustrated in FIGS. 1-7 can also be used here.

The irradiation device of FIG. 11 preferably serves for the post-treatment after the specialist has carried out the first photochemical treatment with short-wavelength light and the radical generating peroxide containing paste (gel, liquid). The patient can also carry out the post-treatment at home by daily irradiating the toenails for about 10-20 minutes with long-wavelength light. Herein, LEDs or arrays having an electric power of 10 watt, e.g. emitting at 630 nm, 670 nm, 740 nm, 850 nm or 940 nm, shortly in the red or near-infrared spectral range, can be used, for example, wherein the beam power density on the nail plate should be larger than 10 mW/cm².

The regular irradiation with light in the red or near-infrared spectral range reaches the fungus spores lying deeper in the tissue which have not been reached in the first photochemical treatment with the short-wavelength radiation because of the lower beam penetration depth. It is observed that the nails regrow clearly and without a fungus infestation by this procedure of a regular irradiation with longer-wavelength light, however only after several months. If ointments or oils with fungicide agents are applied simultaneously with this irradiation and in addition to it to the nail plates and the surrounding tissue, this can only improve and accelerate the healing effect because the light radiation also increases the depth of penetration of the agents into the tissue.

1. Dermatological treatment device for the application in the therapeutical treatment of nail fungus diseases, comprising
a peroxide and/or porphyrine containing substance for the application onto a body area of a patient to be treated, and
an optical irradiation device designed for emitting light onto the body area provided with the substance in a wavelength range between 320 nm and 950nm, preferably between 320 nm and 500 nm and most preferably between 380 nm and 500nm.

2. Dermatological treatment device according to claim 1, wherein the substance comprises H2O2, in particular an aqueous H2O2 solution.

3. Dermatological treatment device according to claim 1, wherein the substance comprises a mixture of an H2O2 solution with glass powder, preferably SiO2 powder and most preferably SiO2 powder with particle sizes in the nanometer range.

4. Dermatological treatment device according to claim 1, wherein the substance further comprises:
a catalyst for accelerating the photochemical H2O2 decay, in particular one or several alkaline hydroxides such as KOH or NaOH and/or NH4 and/or finely grained carbon, and/or
a carrier for increasing the depth of penetration of the substance into the body tissue to be treated, in particular dimethyl sulfoxide or dimethyl sulfone, and/or
a tenside for improving the wettability of the body area to be treated, in particular alkylbenzene sulfonate; cetyl trimethyl ammonium bromide or polyalkylene glycol ether, and/or
a photochemical sensitizer for improving the light effect, in particular dyes such as Eosin Y, Erythrosin or Rose Bengal.

5. Dermatological treatment device according to claim 1, wherein the substance comprises carbamide peroxide.

6. Dermatological treatment device according to claim 1, wherein the substance comprises organic peroxides R—O—O—R, wherein the residues R are equal or unequal and can be H-, alkyl-, aralkyl-, acyl- or aroyl-, e.g. t-butyl hydroperoxide or peroxyacetic acid.

7. Dermatological treatment device according to claim 1, wherein the substance also comprises a gel preferably including sodium polyacrylate.

8. Dermatological treatment device according to claim 1, wherein the substance comprises a mixture of a porphyrine solution and glass powder.

9. Dermatological treatment device according to claim 1, wherein the power value of the light emitted by the optical irradiation device is such that the beam power density at the body area provided with the substance is at least 50 mW/cm2 and at most 300 mW/cm2, preferably more than 75 mW/cm2 and less than 150 mW/cm2.

10. Dermatological treatment device according to claim 1, wherein the optical irradiation device comprises a radiation source in the form of one or several LEDs.

11. Dermatological treatment device according to claim 10, wherein the one or several LEDs are designed to emit light having a wavelength peak between 390 nm and 420 nm, preferably between 395 nm and 415 nm and most preferably at about 400 nm or 405 nm onto the body area provided with the substance.

12. Dermatological treatment device according to claim 10, wherein the one or several LEDs are designed to emit light having a wavelength peak between 350 nm and 380 nm, preferably at about 365 nm and/or between 450 nm and 480 nm, preferably at about 465 nm onto the body area provided with the substance.

13. Dermatological treatment device according to claim 10, wherein the one or several LEDs are designed to emit light having a wavelength peak between 620 nm and 640 nm, 660 nm and 680 nm, 730 nm and 750 nm, 840 nm and 860 nm and/or 930 nm and 950 nm onto the body area to be treated.

14. Dermatological treatment device according to claim 10, wherein the optical irradiation device further comprises a reflector and/or a lens for bundling the radiation emitted by the one or several LEDs into a light stripe or light spot whose beam power density is as homogenous as possible.

15. Dermatological treatment device according to claim 14, wherein the reflector is constructed of two light reflecting plate pairs, the plates of each pair being V-shaped and facing each other opening to the light exit side.

16. Dermatological treatment device according to claim 10, wherein several, in particular four or six, individual diodes in an array are respectively combined to one LED.

17. Dermatological treatment device according to claim 10, wherein the several LEDs are arranged along a straight line.

18. Dermatological treatment device according to claim 10, wherein the several LEDs are arranged at an elongated heat radiator so that LEDs positioned further to the center in the longitudinal direction of the heat radiator are offset transversely to the longitudinal direction with respect to LEDs positioned further outside in the longitudinal direction so that a line connecting an LED positioned outside to an LED positioned further to the center intersects the longitudinal axis at an angle (α) preferably being about 20°.

19. Dermatological treatment device according to one of claim 10, wherein the one or several LEDs are operated by a battery arranged in or at a heat radiator of the irradiation device which is also provided with cooling means.

20. Dermatological treatment device according to claim 1, wherein the optical irradiation device comprises a radiation source in the form of a gas discharge lamp having an elliptoid reflector, in particular an ultra-high pressure Hg lamp, a high-pressure Xe lamp or a tungsten halogen lamp.

21. Dermatological treatment device according to claim 20, wherein the optical irradiation device further comprises a cross-section converter for converting the radiation emitted by the gas discharge lamp to a widened light spot, wherein the cross-section converter is constructed of two internally mirror-coated, triangular metal plates which are mounted almost in parallel to each other, of a triangular thick glass plate having polished surfaces, of a combination of crossed cylindrical lenses of silica glass, of a combination of cylindrical lenses and spherical lenses, of a simple dispersing lens or of a diffuser plate, for example.

22. Dermatological treatment device according to claim 21, wherein the optical irradiation device further comprises a light guide for guiding the light from the gas discharge lamp to the cross-section converter.

23. Dermatological treatment device according to claim 22, wherein the light guide comprises a liquid-filled flexible tube of a fluor-carbon-polymer, preferably Teflon FEP®.

24. Dermatological treatment device according to claim 20, wherein the optical irradiation device comprises an optical multi-filter, in particular a filter wheel by the setting of which the irradiation device is designed to emit light in the full white spectrum excluding the UVA range, i.e. between 400 nm and 800 nm, light in the UVA and blue range, i.e. between 320 nm and 500nm, light in the blue range excluding the UV range, i.e. between 400 nm and 500 nm, light in the UVA range, i.e. between 320 nm and 400 nm, light in the violet range, i.e. between 380 nm and 430 nm, or light in the UVB and UVA and blue range, i.e. between 280 nm and 500 nm, to the body area to be treated.

25. Dermatological treatment device according to claim 1, further comprising an applicator part having a cavity in which the body area to be treated can be accommodated.

26. Dermatological treatment device according to claim 25, wherein the applicator part comprises a shoe-shaped optical shield housing made of a material having the property of a long-pass filter.

27. Dermatological treatment device according to one of the preceding claim 1, wherein the optical irradiation device comprises a funnel or tube shaped added part (87) at its light exit end which is preferably removably pushed onto the light exit end or otherwise attached to it.

28. Dermatological treatment device according to claim 27, wherein the added part
is open at its light exit surface, and/or
is designed to define a distance to the body area to be treated during the treatment, and/or
is made of a material having the property of a long-pass filter.

29. Dermatological treatment device according to claim 26, wherein the long-pass filter is transparent in the long-wavelength portion of the visible spectrum and absorbs in the short-wavelength therapeutic range of the spectrum, i.e. it transmits orange and/or red light and absorbs ultraviolet, blue and green light.

30. Dermatological treatment device according to claim 1, wherein the optical irradiation device comprises a terminating element at its light exit end which is designed to abut plainly against the body area to be treated with its light exit surface during the treatment.

31. Dermatological treatment device according to claim 30, wherein the terminating element
is removably pushed onto the light exit end or otherwise attached to it, and/or
is a cap closed at its light exit surface, and/or
comprises a light exit surface having a plane, concave or convex shape, and/or
is a light exit window attached at a lens tube of the irradiation device or an added part, and/or
is made of a transparent plastic material.

32. Dermatological treatment device according to claim 30, wherein the terminating element or at least its light exit surface is made of Teflon® HP, Dyneon® THV, co-or terpolymers of PTFE, highly transparent or at least translucent and soft silicone® elastomers, highly transparent and soft polyurethane®, PVC® or PE® and/or other carbon-fluor-polymers.

33. Dermatological treatment device according to claim 25, wherein the optical irradiation device is rotatably attached at the applicator part in order to be able to align the emitted light spot to the body area to be treated.

34. Dermatological treatment device according to claim 25, wherein at the light exit end of the optical irradiation device, a plate is attached which is rotatably mounted at the applicator part.

35. Dermatological treatment device according to claim 25, wherein the optical irradiation device is attached at a supporting frame pivotably around a rotation stud, the supporting frame being fixedly mounted at the applicator part.

36. Dermatological treatment device according to claim 1, wherein the treatment device is designed to emit light in the red spectral range, in particular between 600 nm and 700 nm with a wavelength peak at about 630 nm onto the body area to be treated by inserting an insertion plate doped with a red fluorescent dye, in particular Lumogen®, into the beam path of the light emitted by the optical irradiation device.
